(19) United States
(12) Patent Application Publication
(10) Pub. No.: US 2025/0176854 A1
(43) Pub. Date: Jun. 5, 2025

(54) ELECTRONIC DEVICE AND METHOD FOR DETECTING PERIODIC BREATHING

(71) Applicant: Wistron Corporation, New Taipei City (TW)

(72) Inventors: Yu-Hsuan Ho, New Taipei City (TW); Zong Lin Tsai, New Taipei City (TW); Rong Shen Lin, New Taipei City (TW); Shang Min Yeh, New Taipei City (TW); Yu-Wen Huang, New Taipei City (TW)

(73) Assignee: Wistron Corporation, New Taipei City (TW)

(21) Appl. No.: 18/406,189

(22) Filed: Jan. 7, 2024

(30) Foreign Application Priority Data
Dec. 5, 2023 (TW) 112147250

(51) Int. Cl. A61B 5/08; A61B 5/00

(52) U.S. Cl. CPC A61B 5/08; A61B 5/7257; A61B 5/7267

(57) ABSTRACT

An electronic device and a method for detecting a periodic breathing are provided. The method includes: receiving a respiration signal; calculating a variance degree of the respiration signal; performing a first changepoint detection on the variance degree to obtain a first interval; capturing a first interval signal from the respiration signal according to the first interval; detecting the first interval signal to generate a detection result corresponding to at least one periodic breathing; and outputting the detection result.

### CROSS-REFERENCE TO RELATED APPLICATION

This application claims the priority benefit of Taiwan application no. 112147250, filed on Dec. 5, 2023. The entirety of the above-mentioned patent application is hereby incorporated by reference herein and made a part of this specification.

### BACKGROUND

### Technical Field

The disclosure relates to a detection technology, and in particular to an electronic device and a method for detecting a periodic breathing (PB).

### Description of Related Art

With the development of sleep medicine, people are paying more and more attention to sleep quality. There are many products on the market that are used to monitor the sleep state of subjects. Most of the products are used to monitor the sleep state of subjects, rather than the respiration state of subjects. However, the respiration state of the subject is also an important basis for determining the physiological state of subjects. The respiratory pattern of the periodic breathing is that the breathing volume first increases and then decreases, which is an abnormal respiratory pattern. How to detect such abnormal respiratory pattern is one of the important issues in this field.

### SUMMARY

The disclosure provides an electronic device and method for detecting a periodic breathing, which may detect whether a respiratory pattern of a subject is the periodic breathing according to a radar signal.

An embodiment of the disclosure provides an electronic device for detecting a periodic breathing, including a processor and a transceiver. The transceiver receives a respiration signal. The processor is coupled to the transceiver and configured to perform: calculating a variance degree of the respiration signal; performing a first changepoint detection on the variance degree to obtain a first interval; capturing a first interval signal from the respiration signal according to the first interval; detecting the first interval signal to generate a detection result corresponding to at least one periodic breathing; and outputting the detection result through the transceiver.

An embodiment of the disclosure provides a method of detecting the periodic breathing for an electronic device detecting the periodic breathing, including: receiving the respiration signal through the electronic device; calculating the variance degree of the respiration signal; performing the first changepoint detection on the variance degree to obtain the first interval; capturing the first interval signal from the respiration signal according to the first interval; detecting the first interval signal to generate the detection result corresponding to the at least one periodic breathing; and outputting the detection result.

Based on the above, the electronic device of the disclosure may determine whether the subject has the respiratory pattern of the periodic breathing based on a machine learning algorithm or specific rules.

### BRIEF DESCRIPTION OF THE DRAWINGS

FIG. 1 is a schematic diagram of an electronic device for detecting a periodic breathing according to an embodiment of the disclosure.

FIG. 2 is a flowchart of a method for detecting a periodic breathing according to an embodiment of the disclosure.

FIG. 3 is a schematic diagram of a variance signal according to an embodiment of the disclosure.

FIG. 4 is a schematic diagram of a first interval signal according to an embodiment of the disclosure.

FIG. 5 is a schematic diagram of a conversion signal according to an embodiment of the disclosure.

FIG. 6 is a schematic diagram of sampling a second interval signal according to an embodiment of the disclosure.

FIG. 7 is a schematic diagram of a respiration signal of a periodic breathing according to an embodiment of the disclosure.

FIG. 8 is a schematic diagram of a respiration signal of a cheyne-stokes breathing according to an embodiment of the disclosure.

FIG. 9 illustrates a flowchart of a method for detecting a periodic breathing according to an embodiment of the disclosure.

### DESCRIPTION OF THE EMBODIMENTS

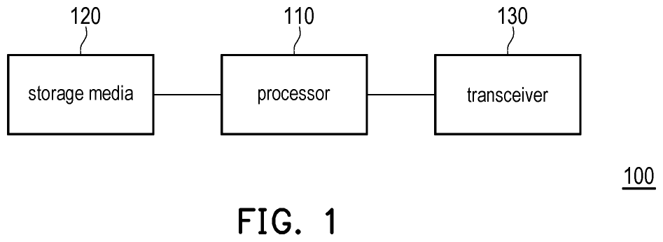

FIG. 1 is a schematic diagram of an electronic device 100 for detecting a periodic breathing according to an embodiment of the disclosure. The electronic device 100 may include a processor 110, a storage media 120, and a transceiver 130.

The processor 110 is, for example, a central processing unit (CPU), or other programmable general-purpose or special-purpose micro control unit (MCU), microprocessor, digital signal processing unit (DSP), programmable controller, application specific integrated circuit (ASIC), graphics processing unit (GPU), image signal processor (ISP)), image processing unit (IPU), arithmetic logic unit (ALU), complex programmable logic device (CPLD), field programmable gate array (FPGA), or other similar components or a combination of the above components. The processor 110 may be coupled to the storage media 120 and the transceiver 130, and access and execute multiple modules and various applications stored in the storage media 120.

The storage media 120 is, for example, any type of fixed or removable random access memory (RAM), read-only memory (ROM), flash memory, hard disk drive (HDD), solid state drive (SSD), or similar components or a combination of the above components, used to store the modules or the various applications that may be executed by the processor 110.

The transceiver 130 transmits or receives signals in a wireless or wired manner. The transceiver 130 may also perform, for example, low noise amplification, impedance matching, mixing, up or down frequency conversion, filtering, amplification, and similar operations.

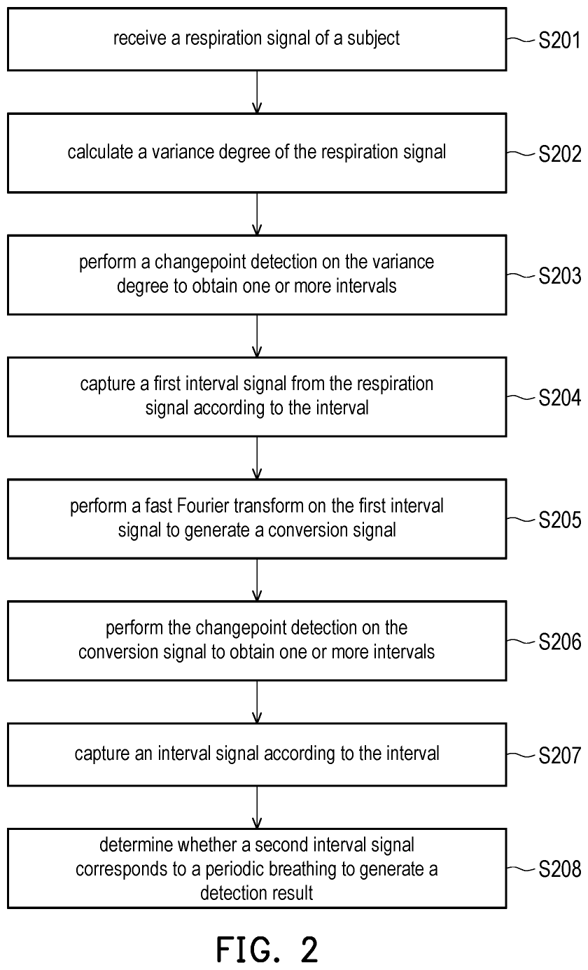

FIG. 2 id a flowchart of a method for detecting a periodic breathing according to an embodiment of the disclosure. The method may be implemented by the electronic device 100 as shown in FIG. 1. The periodic breathing may include, but is not limited to, a general periodic breathing (PB) or a cheyne-stokes breathing (CSB).

In step S201, a respiration signal of a subject may be received by the processor 110 through the transceiver 130. For example, the processor 110 may be communicatively connected to a continuous wave (CW) radar used to measure a respiration of the subject through the transceiver 130, and receive the respiration signal from the CW radar. The CW radar is, for example, a frequency modulated continuous wave (FMCW) radar.

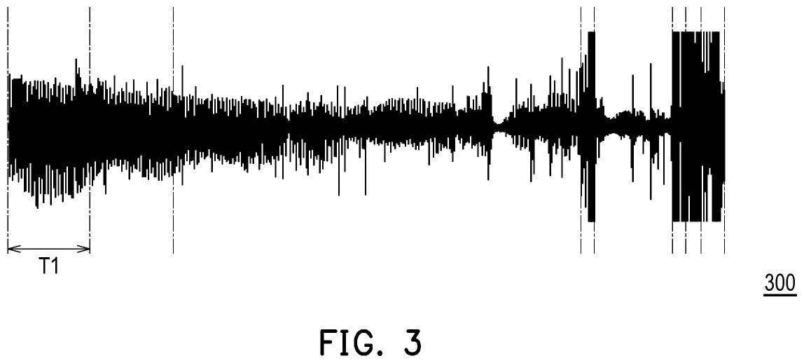

In step S202, a variance degree of the respiration signal may be calculated by the processor 110, such as a variance degree 300 shown in FIG. 3.

In step S203, a changepoint detection (CPD) on the variance degree 300 may be performed by the processor 110 to obtain one or more intervals, such as an interval T1 shown in FIG. 3. Generally speaking, if a sleeping position of the subject does not change, lengths of the various intervals obtained by the processor 110 performing the changepoint detection on the variance degree may be very similar. In an embodiment, before performing the changepoint detection on the variance degree 300, the processor 110 may perform filtering on the variance degree 300. It should be noted that performing filtering on the variance degree 300 by the processor 110 can speed up an entire process operation. However, if the processor 110 does not perform filtering on the variance degree 300, a result after the process operation may be more accurate.

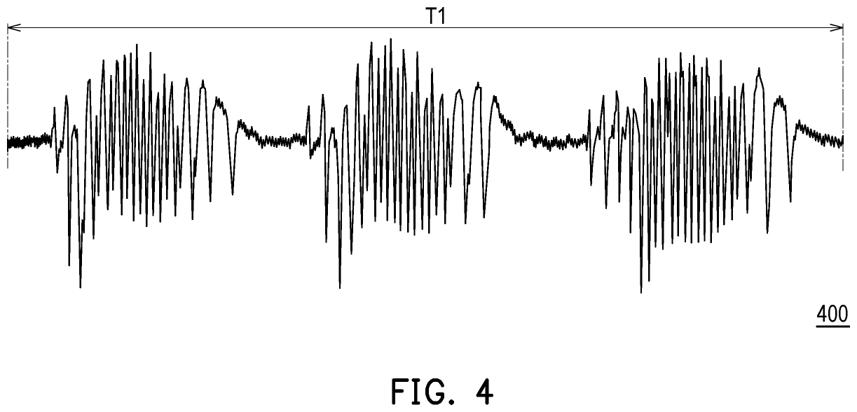

In step S204, a first interval signal may be captured from the respiration signal by the processor 110 according to the interval T1. FIG. 4 is a schematic diagram of a first interval signal 400 according to an embodiment of the disclosure. Referring to FIGS. 3 and 4, the processor 110 may capture the respiration signal during the interval T1 from an original respiration signal as the first interval signal 400. If the subject has a respiratory pattern of the periodic breathing, the first interval signal 400 may be used to generate a detection result corresponding to one or more periodic breathings.

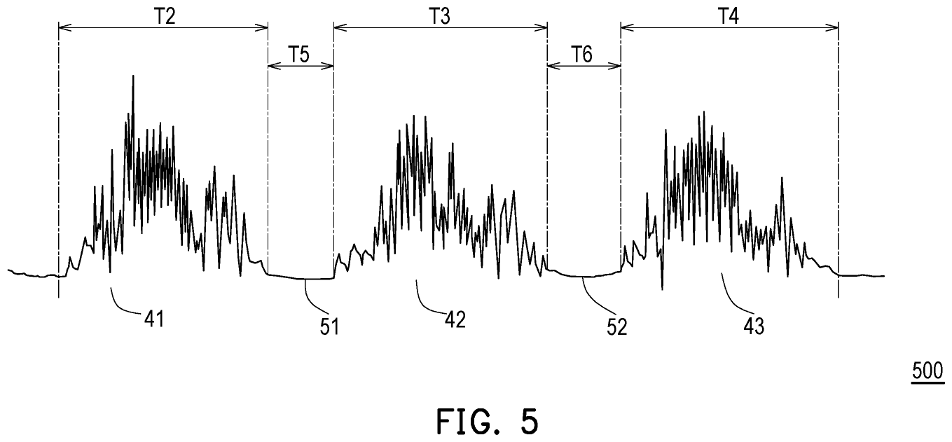

In step S205, a fast Fourier transform (FFT) on the first interval signal 400 may be performed by the processor 110 to generate a conversion signal, such as a conversion signal 500 shown in FIG. 5.

In step S206, the changepoint detection on the conversion signal 500 may be performed by the processor 110 to obtain one or more intervals, such as an interval T2, an interval T3, or an interval T4 as shown in FIG. 5. The interval T2 is adjacent to the interval T3, and the interval T3 is adjacent to the interval T4. The intervals (e.g., the intervals T2, T3, or T4) obtained by the processor 110 in step S206 may be identified as periodic breathing regions (PB regions). In an embodiment, before performing the changepoint detection on the conversion signal 500, the processor 110 may perform filtering on the conversion signal 500. It should be noted that performing filtering on the variance degree 300 by the processor 110 can speed up the entire process operation. However, if the processor 110 does not perform filtering on the variance degree 300, the result after the process operation can be more accurate.

In step S207, an interval signal may be captured from the conversion signal 500 or the respiration signal 400 by the processor 110 according to the interval obtained in step S206 (e.g., the interval T2, T3, or T4).

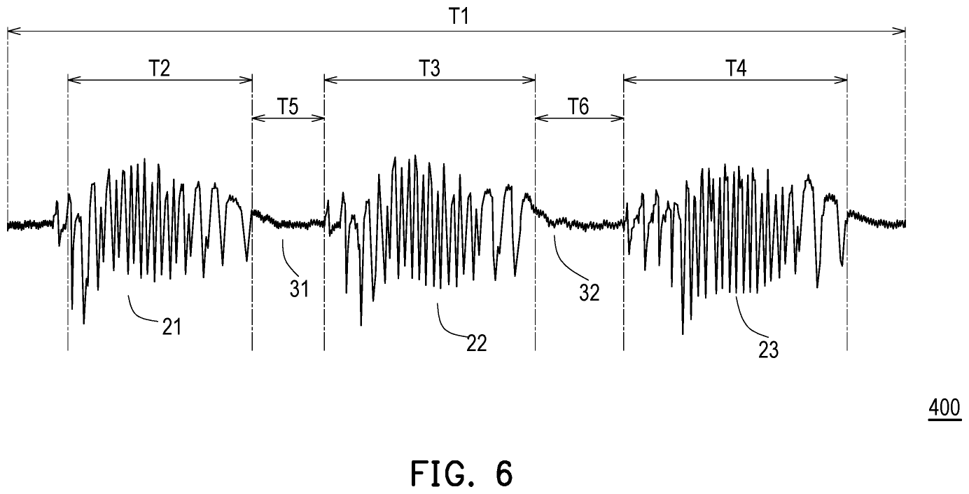

In an embodiment, the interval signal captured in step S207 may include a second interval signal. As shown in FIG. 6, the interval signal may include a second interval signal 21 captured from the respiration signal or the first interval signal 400 according to the interval T2, a second interval signal 22 captured from the respiration signal or the first interval signal 400 according to the interval T3, or a second interval signal 23 captured from the respiration signal or the first interval signal 400 according to the interval T4.

In an embodiment, the interval signal captured in step S207 may include a third interval signal. As shown in FIG. 6, the interval signal may include a third interval signal 31 captured from the respiration signal or the first interval signal 400 according to an interval T5 between the interval T2 and the interval T3 or a third interval signal 32 captured from the respiration signal or the first interval signal 400 according to an interval T6 between the interval T3 and the interval T4. The third interval signal 31 and the third interval signal 32 are equivalent to a front segment signal and a rear segment signal of the second interval signal 22. A starting point of the interval T5 (i.e., the third interval signal 31) may be located at an end point of the interval T2, and the end point of the interval T5 may be located at the starting point of the interval T3. The starting point of the interval T6 (i.e., the third interval signal 32) may be located at the end point of the interval T3, and the end point of the interval T6 may be located at the starting point of the interval T4.

In an embodiment, the interval signal captured in step S207 may include a fourth interval signal. As shown in FIG. 5, the interval signal may include a fourth interval signal 41 captured from the conversion signal 500 according to the interval T2, a fourth interval signal 42 captured from the conversion signal 500 according to the interval T3, or a fourth interval signal 43 captured from the conversion signal 500 according to the interval T4.

In an embodiment, the interval signal captured in step S207 may include a fifth interval signal. As shown in FIG. 5, the interval signal may include a fifth interval signal 51 captured from the conversion signal 500 according to the interval T5 or a fifth interval signal 52 captured from the conversion signal 500 according to the interval T6. The fifth interval signal 51 and the fifth interval signal 52 are equivalent to the front segment signal and the rear segment signal of the fourth interval signal 42. The starting point of the interval T5 (i.e., the fifth interval signal 51) may be located at the end point of the interval T2, and the end point of the interval T5 may be located at the starting point of the interval T3. The starting point of the interval T6 (i.e., the fifth interval signal 52) may be located at the end point of the interval T3, and the end point of the interval T6 may be located at the starting point of the interval T4.

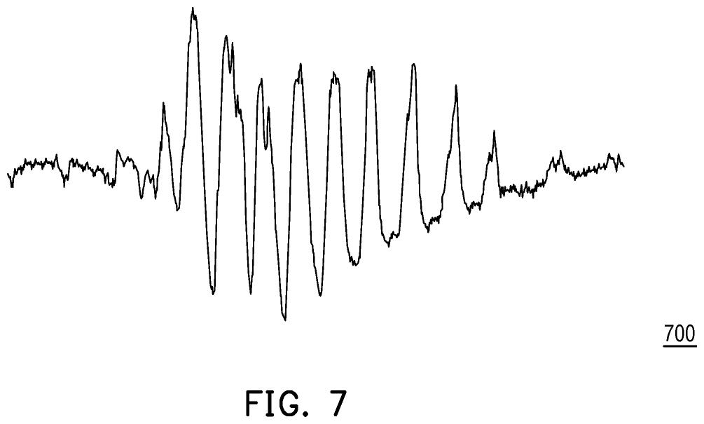

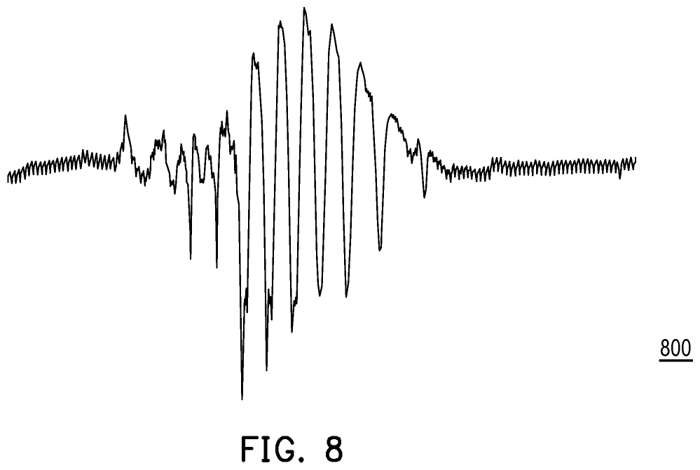

In step S208, whether the second interval signal (for example, the second interval signals 21, 22, or 23) corresponds to the general periodic breathing (a respiration signal 700 as shown in FIG. 7) or the cheyne-strokes breathing (a respiration signal 800 as shown in FIG. 8) may be determined by the processor 110 to generate the detection result. After the detection result is generated, the detection result may be output by the processor 110 through the transceiver 130 for reference by the user of the electronic device 100. In addition to indicating whether the specific second interval signal belongs to the periodic breathing or the cheyne-strokes breathing, the detection result may further include information such as a ratio of the periodic breathing to the sleep time, an average cycle length of the periodic breathing, or a maximum cycle length of the periodic breathing.

In an embodiment, the processor 110 may input the second interval signal to a machine learning model to determine whether the second interval signal corresponds to the periodic breathing. The machine learning model is, for example, a supervised machine learning model.

In an embodiment, the processor 110 may calculate a score corresponding to the second interval signal based on a specific rule and according to a feature value of the second interval signal (or the third interval signal, the fourth interval signal, or the fifth interval signal corresponding to the second interval signal) to determine whether the second interval signal corresponds to the periodic breathing or the cheyne-strokes breathing. Taking the second interval signal 22 as an example, Table 1 is an example of the features of the second interval signal (or the third interval signal, the fourth interval signal, or the fifth interval signal corresponding to the second interval signal), and Table 2 and Table 3 are examples of scoring conditions respectively corresponding to feature 1 and feature 2 in Table 1. The features in Table 1 may include but are not limited to power spectral density (PSD), variance, median, first quartile, third quartile, maximum value, peak width, peak count, skewness, or kurtosis. The processor 110 may determine whether the second interval signal 22 corresponds to the periodic breathing or the cheyne-strokes breathing according to one or more scores respectively come from the second interval signal 22, the third interval signal 31 (or 32), the fourth interval signal 42, or the fifth interval signal 51 (or 52).

TABLE 1

| | third interval | second interval | third interval |
|---|---|---|---|
| | signal 31 | signal 22 | signal 32 |
| | (front segment | (middle | (rear segment |
| | signal) | segment signal) | signal) |
| feature | variance, median, first | power spectral density, | variance, median, first |
| 1 | quartile, third quartile, or | variance, median, first | quartile, third quartile, |
| | maximum value | quartile, third quartile, | or maximum value |
| | | or maximum value | |
| | fifth interval signal | fourth interval | fifth interval signal |
| | 51 (front segment | signal 42 (middle | 52 (rear segment |
| | signal) | segment signal) | signal) |
| feature | variance, median, first | variance, median, first | variance, median, first |
| 2 | quartile, third quartile, or | quartile, third quartile, or | quartile, third quartile, or |
| | maximum value | maximum value, peak | maximum value |
| | | width, peak count, | |
| | | skewness, and kurtosis | |

TABLE 2

| condition | score |
|---|---|
| (third quartile − first quartile of front | −30 |
| segment signal)/(third quartile − first | |
| quartile of middle segment signal) > 0.45 | |
| (third quartile − first quartile of rear | −30 |
| segment signal)/(third quartile − first | |
| quartile of middle segment signal) > 0.45 | |
| variance of front segment signal > | −30 |
| variance of middle segment signal | |
| variance of rear segment signal > | −30 |
| variance of middle segment signal | |
| PSD < 1500 | −15 |
| variance of middle | −25 |
| segment signal < 0.1 | |
| variance of front | −15 |
| segment signal > 0.2 | |
| 0.2 > variance of | −5 |
| front segment signal > 0.1 | |
| variance of front | +10 |
| segment signal < 0.05 | |
| variance of rear | −15 |
| segment signal > 0.2 | |
| 0.2 > variance of | −5 |
| rear segment signal > 0.1 | |
| variance of rear | +10 |
| segment signal < 0.05 | |
| (PSD of middle segment signal of greater | −10 |
| than 0.4 and less than 0.6)/(PSD of middle | |
| segment signal of less than or equal to | |
| 0.4 or greater than or equal to 0.6) > 1.5 | |

TABLE 3

| condition | |
|---|---|
| median of front segment signal > 0.2 | −20 |
| 0.2 > median of middle segment signal > 0.1 | −5 |
| median of rear segment signal > 0.2 | −20 |
| 0.2 > median of rear segment signal > 0.1 | −5 |
| variance of front segment signal > 0.8 | −15 |
| 0.8 > variance of front segment signal > 0.05 | −5 |
| variance of front segment signal < 0.01 | +10 |
| variance of rear segment signal > 0.8 | −15 |
| 0.8 > variance of rear segment signal > 0.05 | −5 |
| variance of rear segment signal < 0.01 | +10 |
| third quartile − first quartile | −10 |
| of front segment signal > 0.5 | |
| third quartile − first quartile | −10 |
| of rear segment signal > 0.5 | |
| maximum value of middle segment signal > 2 | −20 |
| signal of PSD of greater than 0.6 | −20 |
| in middle segment signal < 0.2% | |
| absolute value of skewness > 0.15 | −10 |
| kurtosis > 4 | −10 |
| maximum value of middle segment signal < | −30 |
| maximum value of front segment signal | |
| maximum value of middle segment signal < | −30 |
| maximum value of rear segment signal | |
| signal of PSD of greater than 0.4 in | −10 |
| middle segment signal < 0.22% | |

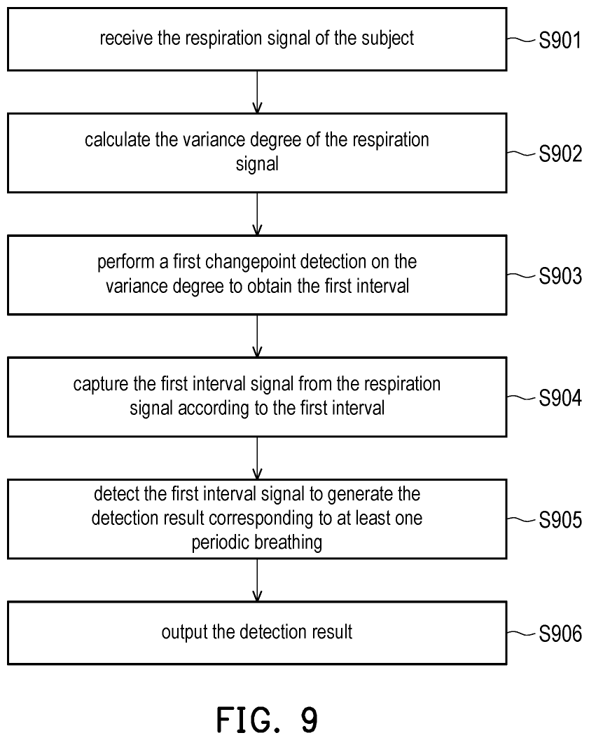

FIG. 9 is a flowchart of a method for detecting a periodic breathing according to an embodiment of the disclosure. The method may be implemented by the electronic device 100 shown in FIG. 1. In step S901, the respiration signal of the subject is received. In step S902, the variance degree of the respiration signal is calculated. In step S903, a first changepoint detection is performed on the variance degree to obtain the first interval. In step S904, the first interval signal is captured from the respiration signal according to the first interval. In step S905, the first interval signal is detected to generate the detection result corresponding to the at least one periodic breathing. In step S906, the detection result is output.

To sum up, the electronic device of the disclosure may obtain the respiration signal of the subject through a non-contact sensor, and may perform signal processing on the respiration signal. The electronic device may detect the processed respiration signal based on a machine learning algorithm or the specific rule to determine whether the respiratory pattern of the subject is the periodic breathing.

What is claimed is:

1. An electronic device for detecting a periodic breathing, comprising:
a transceiver, receiving a respiration signal; and
a processor, coupled to the transceiver and configured to perform:
calculating a variance degree of the respiration signal;
performing a first changepoint detection on the variance degree to obtain a first interval;
capturing a first interval signal from the respiration signal according to the first interval;
detecting the first interval signal to generate a detection result corresponding to at least one periodic breathing; and
outputting the detection result through the transceiver.

2. The electronic device according to claim 1, wherein the processor is further configured to perform:
performing a fast Fourier transform on the first interval signal to generate a conversion signal;
performing a second changepoint detection on the conversion signal to obtain a second interval;
capturing a second interval signal from the respiration signal according to the second interval; and
determining whether the second interval signal corresponds to the periodic breathing to generate the detection result.

3. The electronic device according to claim 2, wherein the processor is further configured to perform:
inputting the second interval signal to a machine learning model to determine whether the second interval signal corresponds to the periodic breathing.

4. The electronic device according to claim 2, wherein the processor is further configured to perform:
determining whether a first feature value of the second interval signal matches a first condition to calculate a first score corresponding to the second interval signal; and
determining whether the second interval signal corresponds to the periodic breathing according to the first score.

5. The electronic device according to claim 4, wherein the first feature value is associated with one of the following features: power spectral density, variance, median, first quartile, third quartile, and maximum value.

6. The electronic device according to claim 4, wherein the processor is further configured to perform:
performing the second changepoint detection on the conversion signal to obtain a third interval adjacent to the second interval;
capturing a third interval signal from the respiration signal according to the second interval and the third interval; and
determining whether a second feature value of the third interval signal matches a second condition to calculate the first score.

7. The electronic device according to claim 6, wherein the second feature value is associated with one of the following features: variance, median, first quartile, third quartile, and maximum value.

8. The electronic device according to claim 4, wherein the processor is further configured to perform:
capturing a fourth interval signal from the conversion signal according to the second interval;
determining whether a second feature value of the fourth interval signal matches a second condition to calculate a second score corresponding to the fourth interval signal; and
determining whether the second interval signal corresponds to the periodic breathing according to the first score and the second score.

9. The electronic device according to claim 8, wherein the second feature value is associated with one of the following features: variance, median, first quartile, third quartile, maximum value, peak width, peak count, skewness, and kurtosis.

10. The electronic device according to claim 8, wherein the processor is further configured to perform:
performing the second changepoint detection on the conversion signal to obtain a third interval adjacent to the second interval;
capturing a fifth interval signal from the conversion signal according to the second interval and the third interval; and
determining whether a third feature value of the fifth interval signal matches a third condition to calculate the second score.

11. The electronic device according to claim 10, wherein the third feature value is associated with one of the following features: variance, median, first quartile, third quartile, and maximum value.

12. A method of detecting a periodic breathing for an electronic device detecting the periodic breathing, comprising:
receiving a respiration signal through the electronic device;
calculating variance of the respiration signal to generate a variance degree;
performing a first changepoint detection on the variance degree to obtain a first interval;
capturing a first interval signal from the respiration signal according to the first interval;
detecting the first interval signal to generate a detection result corresponding to at least one periodic breathing; and
outputting the detection result.

13. The method according to claim 12, wherein detecting the first interval signal to generate the detection result corresponding to the at least one periodic breathing comprises:
performing a fast Fourier transform on the first interval signal to generate a conversion signal;
performing a second changepoint detection on the conversion signal to obtain a second interval;
capturing a second interval signal from the respiration signal according to the second interval; and
determining whether the second interval signal corresponds to a periodic breathing to generate the detection result.

14. The method according to claim 13, wherein determining whether the second interval signal corresponds to the periodic breathing to generate the detection result comprises:
inputting the second interval signal to a machine learning model to determine whether the second interval signal corresponds to the periodic breathing.

15. The method according to claim 13, wherein determining whether the second interval signal corresponds to the periodic breathing to generate the detection result comprises:
determining whether a first feature value of the second interval signal matches a first condition to calculate a first score corresponding to the second interval signal; and
determining whether the second interval signal corresponds to the periodic breathing according to the first score.

16. The method according to claim 15, wherein the first feature value is associated with one of the following features: power spectral density, variance, median, first quartile, third quartile, and maximum value.

17. The method according to claim 15, wherein determining whether the first feature value of the second interval signal matches the first condition to calculate the first score corresponding to the second interval signal comprises:
performing the second changepoint detection on the conversion signal to obtain a third interval adjacent to the second interval;
capturing a third interval signal from the respiration signal according to the second interval and the third interval; and
determining whether a second feature value of the third interval signal matches a second condition to calculate the first score.

18. The method according to claim 17, wherein the second feature value is associated with one of the following features: variance, median, first quartile, third quartile, and maximum value.

19. The method according to claim 15, wherein determining whether the second interval signal corresponds to the periodic breathing according to the first score comprises:
capturing a fourth interval signal from the conversion signal according to the second interval;
determining whether a second feature value of the fourth interval signal matches a second condition to calculate a second score corresponding to the fourth interval signal; and
determining whether the second interval signal corresponds to the periodic breathing according to the first score and the second score.

20. The method according to claim 19, wherein the second feature value is associated with one of the following features: variance, median, first quartile, third quartile, maximum value, peak width, peak count, skewness, and kurtosis.

21. The method according to claim 19, further comprising:
performing the second changepoint detection on the conversion signal to obtain a third interval adjacent to the second interval;
capturing a fifth interval signal from the conversion signal according to the second interval and the third interval; and
determining whether a third feature value of the fifth interval signal matches a third condition to calculate the second score.

22. The method according to claim 21, wherein the third feature value is associated with one of the following features: variance, median, first quartile, third quartile, and maximum value.
